(19) United States
(12) Patent Application Publication
(10) Pub. No.: US 2015/0374248 A1
(43) Pub. Date: Dec. 31, 2015

(54) DEVICE AND METHOD FOR MEASURING BLOOD PRESSURE

(71) Applicant: PROLIFIC TECHNOLOGY INC., Taipei (TW)

(72) Inventors: Weichih Hu, Taipei (TW); Cheng-Sheng Chan, Taipei (TW); Ming-Cheng Chang, Taipei (TW)

(21) Appl. No.: 14/317,116

(22) Filed: Jun. 27, 2014

(51) Int. Cl. A61B 5/022; A61B 5/00

(52) U.S. Cl. CPC A61B 5/02225; A61B 5/02233; A61B 5/749; A61B 5/742

(57) ABSTRACT

According to one embodiment of a device for measuring blood pressure, the device includes a pressure sensor, a microprocessor, and a user interface, wherein a user exerts pressure on the user's wrist by using the pressure sensor, the pressure sensor senses the pressure to produce oscillation signal, the microprocessor connects with the pressure sensor and receives the oscillation signal to calculate vessel pulse, systolic blood pressure, and diastolic blood pressure of the user, the user interface connects with the microprocessor and receives instruction data of the microprocessor to inform the user.

### BACKGROUND

Recently, necessary configuration for measuring blood pressure, either on arm, on wrist, or through tunnel, uses a pump to push the air into the cuff for compressing blood vessel of upper arm, until blood flow stops to begin discouraging pressure for measurements. FIG. 1 illustrates a typical block diagram for measuring blood pressure. In FIG. 1, the cuff is inflatable to be used to cover arm or wrist. The pump with the driver circuit inflates the cuff, allowing the cuff oppressive to arm or wrist so that the blood vessels can not flow. Slow discouraged valve allows pressure sensor circuit to detect cuff pressure and pulse waveform of the blood pressure. The microprocessor (MCU) is used to control the operation of the entire sphygmomanometer, including inflates pump, performs slow discourage, converts pressure values, calculates vessel pulse, diastolic blood pressure, and systolic blood pressure, and handles the user interface such as buttons, display, and voice indication, etc. This way of blood pressure measurement needs performing inflation and deflation of the cuff that clad arm or wrist, and this process takes a period of time. But with different feeling on the pressure, most users have tightened uncomfortable feeling when the sphygmomanometers start increasing pressure till discourage. Another problem is the process is not fast that the time required for increasing pressure and discourage takes several tens of seconds. Furthermore the apparatus for measuring blood pressure usually needs pneumatic pump motor for inflation and deflation of the cuff, thus the cost is extremely impressive.

The above mentioned blood pressure measurement technique requires many configurations and time-consuming process, so that the cost is relatively high. To improve the shortcomings of using inflatable pump motor and cuff for time-consuming blood pressure measurements, the present invention provides a blood pressure measurement technique to make blood pressure measurement simply and quickly, and easily to be carried.

### SUMMARY

The exemplary embodiments of the disclosure may provide a device and method for measuring blood pressure.

One exemplary embodiment relates to a device for measuring blood pressure, the device includes a pressure sensor, a microprocessor, and a user interface, wherein a user exerts pressure on the user's wrist by using the pressure sensor, the pressure sensor senses the pressure to produce oscillation signal; the microprocessor connects with the pressure sensor and receives the oscillation signal to calculate vessel pulse, systolic blood pressure, and diastolic blood pressure of the user, the user interface connects with the microprocessor and receives data of the microprocessor to inform the user.

Another exemplary embodiment relates to a method of measuring blood pressure, adapted to a pressure sensor, a microprocessor, and a user interface, the method includes: a user exerts pressure on user's wrist by using the pressure sensor for sensing the pressure to produce oscillation signal; uses the microprocessor to calculate vessel pulse, systolic blood pressure, and diastolic blood pressure of the user through the oscillation signal; uses the user interface to inform the user according to instruction data of the microprocessor.

### BRIEF DESCRIPTION OF THE DRAWINGS

FIG. 1 illustrates a typical block diagram for measuring blood pressure.

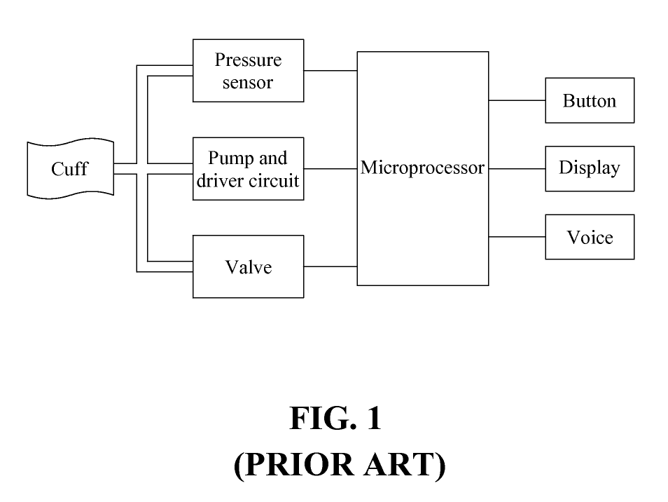

FIG. 2 illustrates a device for measuring blood pressure, according to an exemplary embodiment.

FIG. 3 illustrates method of using interpolation in the portion of pressure waveform to estimate complete waveform, according to an exemplary embodiment.

FIG. 4 illustrates a flowchart of the device for measuring blood pressure in FIG. 2, according to an exemplary embodiment.

FIG. 5a illustrates an external view of the device for measuring blood pressure, according to an exemplary embodiment.

FIG. 5b illustrates another external view of the device for measuring blood pressure, according to an exemplary embodiment.

FIG. 6 illustrates a method for measuring blood pressure, according to an exemplary embodiment.

### DETAILED DESCRIPTION OF DISCLOSED EMBODIMENTS

Below, exemplary embodiments will be described in detail with reference to accompanying drawings so as to be easily realized by a person having ordinary knowledge in the art. The inventive concept may be embodied in various forms without being limited to the exemplary embodiments set forth herein. Descriptions of well-known parts are omitted for clarity, and like reference numerals refer to like elements throughout.

The exemplary embodiments in the disclosure provide a blood pressure measurement technique. More particularly, the disclosure relates to a blood pressure measurement technique with quick operation and low cost. The blood pressure measurement technique of the disclosure utilizes a new mechanism of calculating the blood pressure such that cuff and associated components such as valves and pumps may be omitted. This technique may measure blood pressure of power saving and lower cost.

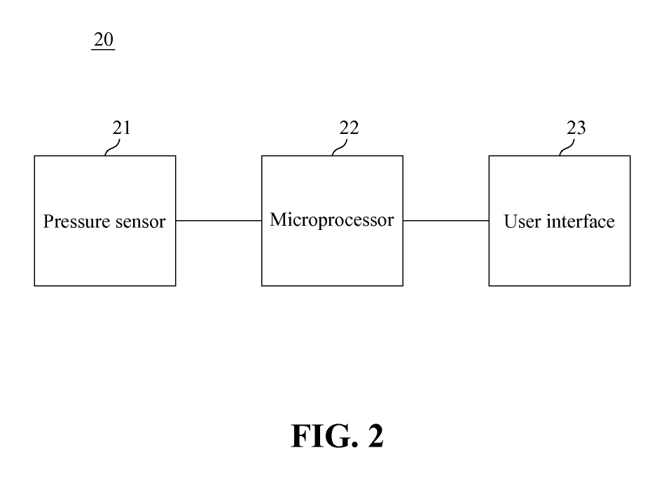

FIG. 2 illustrates a device for measuring blood pressure, according to an exemplary embodiment. Refer to FIG. 2, the device for measuring blood pressure 20 includes a pressure sensor 21, a microprocessor 22, and a user interface 23, wherein a user exerts pressure on user's wrist by using the pressure sensor 21, the pressure sensor 21 senses the pressure to produce oscillation signal; the microprocessor 22 connects with the pressure sensor 21, and receives the oscillation signal to calculate pulse, systolic blood pressure, and diastolic blood pressure of the user; the user interface 23 connects with the microprocessor 22, and receives instruction data of the microprocessor to inform the user.

In FIG. 2, a user exerts pressure on the user's wrist by using the pressure sensor 21, the pressure sensor 21 senses the pressure to produce oscillation signal. The user must make pressure sensor contact with the user's wrist to sense the pulse signal. If the pulse signal is not sensed by the pressure sensor, i.e., the microprocessor 22 receives no oscillation signal, then the microprocessor 22 transmits instruction data to the user interface 23 to inform the user to make position adjustment, such as the user moves the pressure sensor 21 to the position of pulse signal can be sensed. This instruction data is such as voice, light, or text/numeric display and so on.

Following the above mentioned, the user exerts pressures on the user's wrist by using pressure sensor 21 for performing measurement, if the exerted pressure is inadequate or insufficient, the microprocessor 22 transmits instruction data to the user interface 23 to inform the user to make adjustment. As mentioned above, the microprocessor transmits instruction data such as sound, flash light, or text/numeric display to inform user to perform exerting pressure or relaxing pressure.

When a user applying pressure on top of the blood vessels by using the pressure sensor 21, pressure sensors 21 would sense consolidated pressure waveform, i.e., the pressure that the user exerts on the blood vessels and the rebound pressure of vessel pulse. Thus the pressure sensor 21 may sense the oscillation signal of the vessel pulse. The microprocessor 22 is able to get enough pressure readings and enough pressure waveform via user repeatedly performs exerting pressure or relaxing pressure by using the pressure sensor 21. The microprocessor 22 then reorders sufficient pressure waveforms to estimate completed pressure waveform, this estimation means is such as using internal and/or external interpolation.

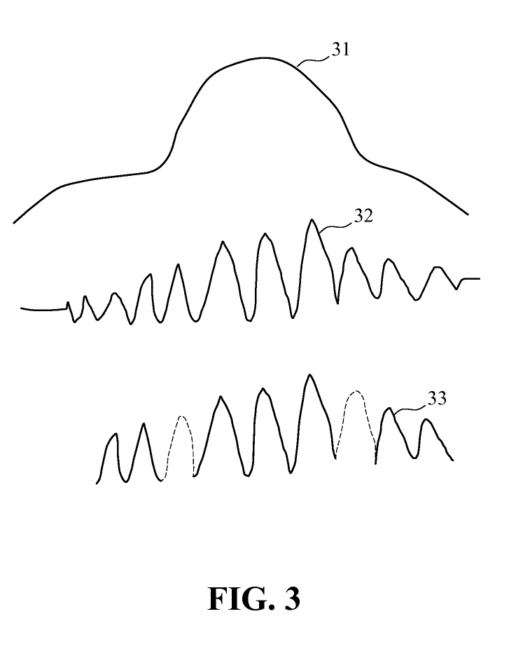

FIG. 3 illustrates method of using interpolation in the portion of pressure waveform to estimate complete waveform, according to an exemplary embodiment. Refer to FIG. 3, the microprocessor 22 uses the filter to separate the received oscillation signal into the pressure waveform 31 and the pulse waveform 32. Then the microprocessor 22 reorders the pressure waveform based on the pressure exerted by the user, and performs interpolation for the pressure waveform (dotted line waveform) to estimate the completed pressure waveform 33. Finally, the microprocessor 22 calculates vessel pulse, systolic blood pressure, and diastolic blood pressure according to this complete pressure waveform 33, by using such as oscillometric method. The microprocessor 22 may also transmits calculated vessel pulse, systolic blood pressure, and diastolic blood pressure to the user interface 23 such as a numeric display, and transmits instruction of measurement is completed to the user interface 23 such as voice.

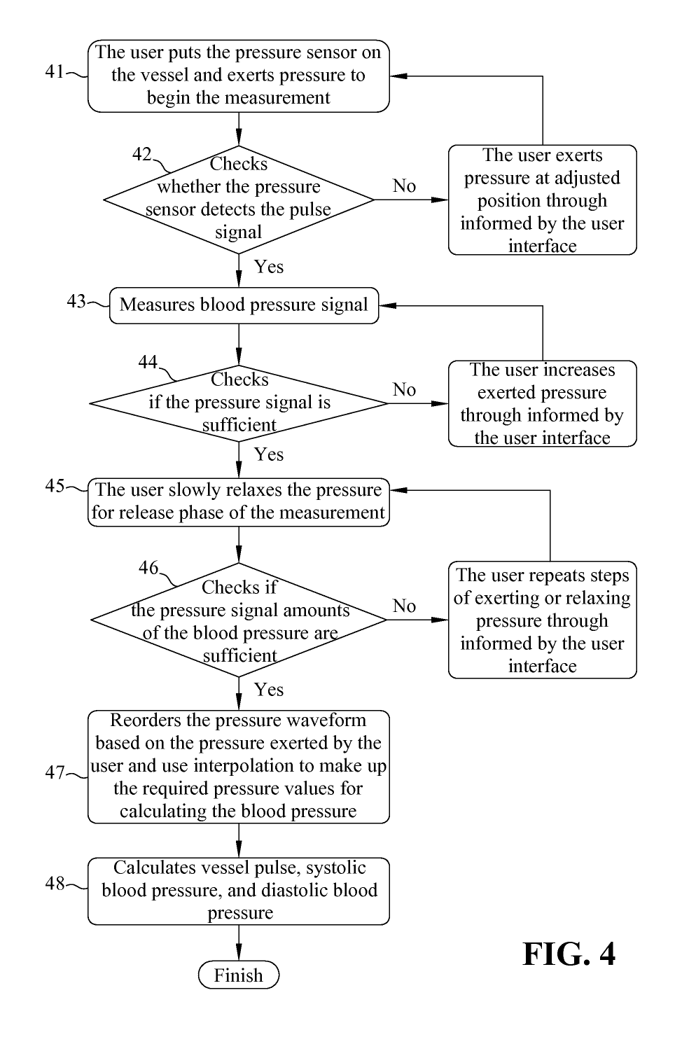

The procedure of above mentioned blood pressure measurement may refer to FIG. 4. FIG. 4 illustrates a flowchart of the device for measuring blood pressure in FIG. 2, according to an exemplary embodiment. In FIG. 4, first the user puts the pressure sensor on the vessel and exerts pressure to begin the measurement (step 41); checks whether the pressure sensor detects the pulse signal (step 42); If not, then the user exerts pressure at adjusted position through informed by the user interface (backs to step 41); then, measures blood pressure signal through the pressure sensor (step 43); checks if the pressure signal is sufficient (step 44); If not, then the user increases exerted pressure through informed by the user interface (backs to step 43); then the user slowly relaxes the pressure for release phase of the measurement (step 45); checks if the pressure signal amounts of the blood pressure are sufficient (step 46); if not, the user repeats steps of exerting or relaxing pressure through informed by the user interface (return to step 45); reorders the pressure waveform based on the pressure exerted by the user and use interpolation to make up the required pressure values for calculating the blood pressure (step 47); finally calculates vessel pulse, systolic blood pressure, and diastolic blood pressure (step 48).

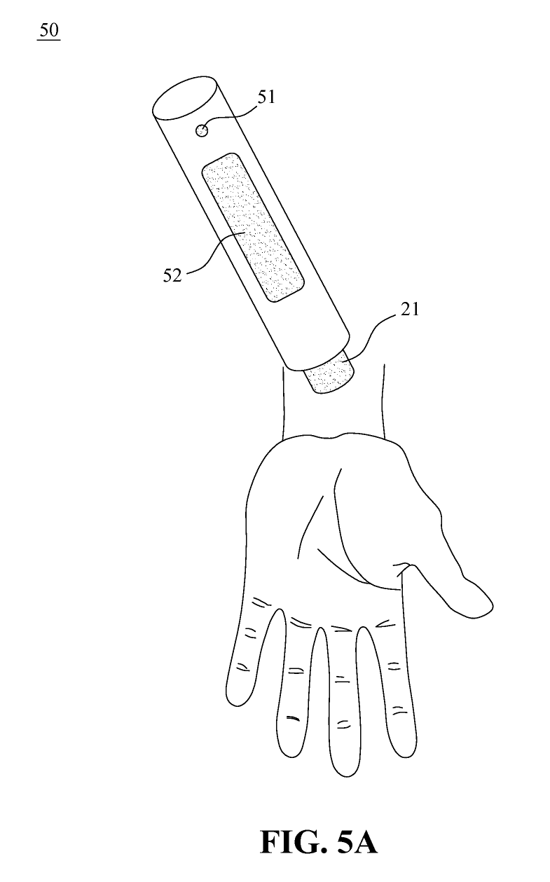

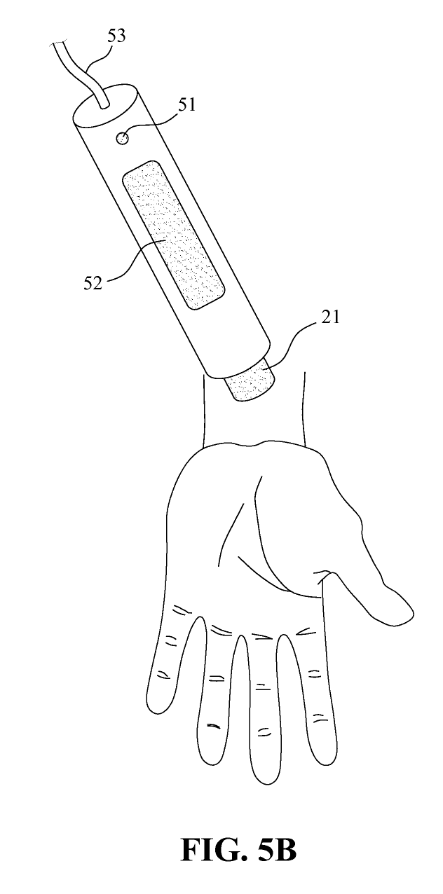

There are many implementations with a variety of exterior style for using the device of blood pressure measurement in FIG. 2 with the measurement procedure in FIG. 4. FIG. 5a illustrates an external view of the device for measuring blood pressure, according to an exemplary embodiment. Refer to FIG. 5, a user exerts pressure on user's wrist by using a pressure sensor 21 via a pen-style sphygmomanometer 50. The microprocessor 22 transmits operation instructions to the user interface 23 represented by the light 51. The microprocessor 22 transmits instructions of exerting pressure or relaxing pressure to the user interface 23 to inform the user making adjustment via voice. Finally, the microprocessor 22 may transmit systolic pressure, diastolic pressure, and vessel pulse to the user interface 23, indicated by a numeric display 52.

In FIG. 5a, the power supply for the device of blood measurement is a built-in battery power supply. Another way of power supply may use the AC power supply and power converter, or use a USB host to supply power. This way of external power supply is such as shown in FIG. 5b. In FIG. 5b, an external power supply cord 53 uses AC power cord or USB cable to connect the power supply. And systolic blood pressure, diastolic blood pressure, and vessel pulse may also be transmitted to the USB host via USB cable. The USB host is such as PC, tablet, or smart phone. In FIG. 5b, the USB cable may also be used to transmit digitized oscillation signal from the microprocessor to the USB host such that the USB host may calculate systolic blood pressure, diastolic blood pressure, and vessel pulse, i.e., the calculation is performed by the USB host instead of the microprocessor.

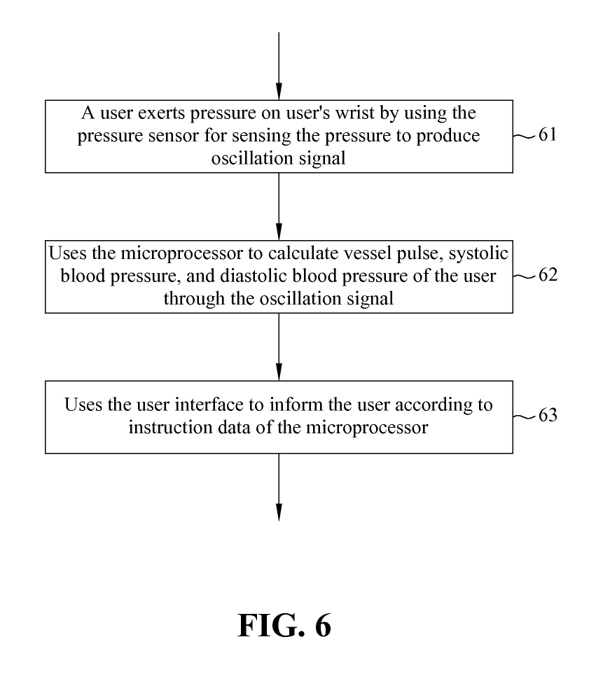

FIG. 6 illustrates a method for measuring blood pressure, according to an exemplary embodiment. Refer to FIG. 6, this method for measuring blood pressure is adapted to a pressure sensor, a microprocessor, and a user interface, the method includes: a user exerts pressure on user's wrist by using the pressure sensor for sensing the pressure to produce oscillation signal (step 61); uses the microprocessor to calculate vessel pulse, systolic blood pressure, and diastolic blood pressure of the user through the oscillation signal(step 62); uses the user interface to inform the user according to instruction data of the microprocessor (step 63).

As mentioned above, the instruction data of the microprocessor in FIG. 6 may include operation instruction, position adjustment, exerting pressure or relaxing pressure, the measured values, and measurement is completed, and so on. And the user interface may be one or any combination of voice, light, or text/numeric display.

In summary, the blood pressure measurement technique uses pressure sensor directly applying pressure on the blood vessel and calculation mechanism with interpolation of pressure waveform, so that cuff, pump, and valve are omitted. Therefore, blood pressure measurement technique of the present invention is a blood pressure measurement technology of quick operation and cost reduction.

It will be apparent to those skilled in the art that various modifications and variations can be made to the disclosed embodiments. It is intended that the specification and examples be considered as exemplary only, with a true scope of the disclosure being indicated by the following claims and their equivalents.

What is claimed is:

1. A device for measuring blood pressure comprising:
a pressure sensor;
a microprocessor; and
a user interface;
wherein a user exerts pressure on said user's wrist by using said pressure sensor, said pressure sensor senses said pressure to produce oscillation signal; said microprocessor connects with said pressure sensor, and receives said oscillation signal to calculate vessel pulse, systolic blood pressure, and diastolic blood pressure of said user; said user interface connects with said microprocessor and receives instruction data of said microprocessor to inform said user.

2. The device as claimed in claim 1, wherein said microprocessor uses filter to separate said received oscillation signal into pressure waveform and pulse waveform, and said microprocessor reorders said pressure waveform based on said pressure exerted by said user and performs interpolation for said pressure waveform to estimate completed said pressure waveform.

3. The device as claimed in claim 1, wherein said microprocessor uses oscillometric method to calculate vessel pulse, systolic blood pressure, and diastolic blood pressure of said user.

4. The device as claimed in claim 1, wherein said instruction data includes operation instruction, position adjustment, exerting pressure or relaxing pressure, measured values, and measurement is completed.

5. The device as claimed in claim 1, wherein said user interface is combination of voice, light, or text/numeric display.

6. A method for measuring blood pressure, adapted to a pressure sensor, a microprocessor, and a user interface, the method comprising:
a user exerts pressure on said user's wrist by using said pressure sensor for sensing pressure to produce oscillation signal;
uses said microprocessor to calculate vessel pulse, systolic blood pressure, and diastolic blood pressure of said user through said oscillation signal;
uses said user interface to inform said user according to instruction data of said microprocessor.

7. The method as claimed in claim 6, wherein said microprocessor uses filter to separate said received oscillation signal into pressure waveform and pulse waveform, and said microprocessor reorders said pressure waveform based on said pressure exerted by said user and performs interpolation for said pressure waveform to estimate completed said pressure waveform.

8. The method as claimed in claim 6, wherein said microprocessor uses oscillometric method to calculate vessel pulse, systolic blood pressure, and diastolic blood pressure of said user.

9. The method as claimed in claim 6, wherein said instruction data includes operation instruction, position adjustment, exerting pressure or relaxing pressure, measured values, and measurement is completed.

10. The method as claimed in claim 6, wherein said user interface is combination of voice, light, or text/numeric display.
